(19) United States
(12) Patent Application Publication
(10) Pub. No.: US 2026/0026822 A1
(43) Pub. Date: Jan. 29, 2026

(54) DEPTH SENSING BONE DRILL AND METHOD OF USE

(71) Applicant: Ryan Danner, Somersworth, NH (US)

(72) Inventor: Ryan Danner, Somersworth, NH (US)

(21) Appl. No.: 19/283,117

(22) Filed: Jul. 28, 2025

Related U.S. Application Data
(60) Provisional application No. 63/676,881, filed on Jul. 29, 2024.
(60) Provisional application No. 63/775,342, filed on Mar. 21, 2025.

(51) Int. Cl. A61B 17/16; A61B 17/00; A61B 17/56; A61B 90/00

(52) U.S. Cl. CPC A61B 17/162; A61B 17/1624; A61B 17/1628; A61B 17/56; A61B 90/06; A61B 2017/00022; A61B 2017/00199; A61B 2017/00734; A61B 2017/564; A61B 2090/061; A61B 2090/062; A61B 2090/064

(57) ABSTRACT

A depth sensing drill bit chuck can measure the depth of a drilled hole in real time as a hole is being drilled. A depth sensing drill bit chuck can use speed and/or force measurements to determine the moment a drill bit tip passes out of a target object, and the depth sensing drill bit chuck can determine the hole depth at the moment the drill bit tip passes out of the target object.

### RELATED APPLICATIONS

This application claims the benefit of U.S. Provisional Application Ser. No. 63/676,881, filed Jul. 29, 2024, entitled DEPTH SENSING BONE DRILL AND METHOD OF USE, and the benefit of U.S. Provisional Application Ser. No. 63/775,342, filed Mar. 21, 2025, entitled DEPTH SENSING BONE DRILL AND METHOD OF USE, the entire disclosures of which are herein incorporated by reference.

### FIELD OF THE INVENTION

This application relates to relates to the field of medical apparatus/devices, and more particularly, the present invention relates to a hand-held bone drilling device facilitating drilling of a screw into a bone in a precise manner and with a precise depth.

### BACKGROUND OF THE INVENTION

In various surgeries such as fixation of broken bones, screws and plates may be used to hold bone fragments together and facilitate healing. Screwing a plate to a bone requires that a surgeon first drill a hole in the bone so that the bone can accommodate the screw. This is typically accomplished using a specialized bone drill. In many cases, the hole will extend the entire way through the bone from one side to the other so that the screw can be in contact with the most bone possible to create the most strength.

An ideal hole will accommodate a screw that extends from one side to the other, through as much of the bone as possible. However, it can be very difficult for the surgeon to drill a hole to the best depth from one side to the other. Many surgeons attempt to drill holes based on a sense of feeling and intuition, often with mixed results.

Selecting the ideal length of screw that best fits through the bone from one side to the other can be another challenge. If a screw is too short, the screw will have poor purchase and may not hold stably. Even worse, if the screw is too long and protrudes out from the other side of the bone, the patient can be subjected to tissue irritation, tendon rupture, or other serious injury. Furthermore, if the surgeon is unable to select and use the correct length screw in the first try, repeated attempts can compromise the bone resulting in poor purchase and instable fixation.

The traditional method includes the use of a depth gauge to determine the best length for a screw to use all of the available depth without extending out the other side of the hole. However, depth gauges have proven difficult to use, and often have accuracy rates of less than 50%, according to some studies. In addition to the human toll, repeated attempts can be costly in an operating room that often costs hundreds of dollars or more per minute.

It would be desirable to have a system and method that allows for the bone to be drilled to an optimal depth and for that depth to be easily determined so that an optimal length screw can then be selected and used. It would be further desirable if the system and method can be used quickly so that time can be saved compared with traditional systems and methods.

### SUMMARY OF THE INVENTION

The system and method described herein overcome disadvantages of the prior art by quickly drilling to a correct depth in a bone and selecting the correct screw size that maximizes purchase while avoiding damage from extending too far through the other side of the bone. The description herein furthermore overcomes disadvantages of the prior art by saving time without the need for the additional step of using a depth gauge and without the need for multiple attempts. This description overcomes disadvantages of the prior art by quickly and accurately determining the drill bit depth, also referred to as the penetration depth, when the drill bit penetrates the far cortex, which can also be referred as the distant side of the bone. That is to say, the depth of the hole and the ideal screw length can be determined as the drill bit reaches the distant side of the bone. This allows the optimal screw length to be quickly selected the first time, providing the best purchase in the surrounding bone and without the screw protruding through the distant side of the bone.

In an embodiment, a depth sensing drill chuck can include a drill bit securing area adapted to secure a drill bit, at least one range finder adapted to measure a distance between the depth sensing drill chuck and a target surface, and a processor that can use the measured distance between the depth sensing drill chuck and the target surface to calculate a penetration depth in real time as the target surface is being penetrated.

The depth sensing drill chuck can include a stationary portion adapted to be engaged with a body of drill base and a rotary portion adapted to be operatively engaged with a motor of the drill base, the depth sensing drill chuck further comprising an electricity-generating dynamo between the stationary portion and the rotary portion. The depth sensing drill chuck can include a battery adapted to maintain an uninterrupted power supply to the range finder and the processor after the rotary portion has stopped spinning. The depth sensing drill chuck can include a load cell adapted to measure force applied to the drill bit as the drill bit penetrates the target surface, wherein the processor uses the measured distance and the measured force to calculate a penetration depth in real time as the target surface is being penetrated. The depth sensing drill chuck can include a laser aiming motor that keeps the laser focused on a focus point on the target surface as the depth sensing drill chuck moves closer to the target surface during drilling. The range finder can be located on the rotary portion so that the distance between the depth sensing drill chuck and the target surface can be measured at multiple locations around the 360-degree rotary path. The processor can have a measured distance averaging module that averages the measured distances at multiple rotary positions to calculate an averaged measured distance that can be used as the measured distance to calculate the penetration depth. The processor can include a speed calculation module that can monitor the change in penetration depth over time to determine a speed at which the target surface is being penetrated, and wherein the processor further comprises a screw length determining module adapted to use a change in speed to determine the moment that the drill bit tip passes out of a second cortex layer of a bone.

A depth sensing drill chuck can include at least one range finder adapted to measure a distance between the depth sensing drill chuck and a target surface, and a load cell adapted to measure force applied to a drill bit as the drill bit penetrates a target surface. The depth sensing drill chuck can include a stationary portion adapted to be engaged with a body of drill base and a rotary portion adapted to be operatively engaged with a motor of the drill base, the depth sensing drill chuck can include an electricity-generating dynamo between the stationary portion and the rotary portion. The depth sensing drill chuck can include a battery adapted to maintain an uninterrupted power supply to the range finder and the load cell after the rotary portion has stopped spinning. The depth sensing drill chuck can include a laser aiming motor that keeps the at least one laser focused on a focus spot on the target surface as the depth sensing drill chuck moves closer to the target surface during drilling. The at least one range finder can include two or more range finders, and wherein the two or more range finders measure the distance between the depth sensing drill chuck and the target surface to generate multiple measured distances, and wherein the multiple measured distances are averaged to overcome variances due to uneven variations in the target surface or due to changes in the rotary position of a drill body. The depth sensing drill chuck can include two or more laser aiming motors that keeps the two or more lasers focused on two or more focus spots on the target surface as the depth sensing drill chuck moves closer to the target surface during drilling. A range finder is located on the rotary portion so that the distance between the depth sensing drill chuck and the target surface can be measured at multiple locations around the 360-degree rotary path. The depth sensing drill chuck can include a laser aiming motor that keeps the laser focused on a focus spot on the target surface as the depth sensing drill chuck moves closer to the target surface during drilling.

A method of fixing broken bones can include drilling a hole in a bone using a depth measuring drill chuck, the depth measuring drill chuck comprising a load cell and at least one range finder adapted to measure a distance between the depth sensing drill chuck and the broken bone, the depth measuring drill chuck calculating a penetration depth in real time while drilling the hole, and reading the penetration depth from a display screen.

Reading the penetration depth from the display screen can include reading the penetration depth from the display screen in real time, while drilling. The method can include selecting a fixing screw with a length that matches the penetration depth, and further comprising screwing the fixing screw into the hole, wherein the screw length is the same as the hole depth.

### BRIEF DESCRIPTION OF THE DRAWINGS

The invention description below refers to the accompanying drawings, of which:

FIG. 1 is a perspective view of a depth sensing drill system, according to an illustrative embodiment;

FIG. 2A is a cross sectional view of a depth sensing chuck, taken along cross section line 2-2 of FIG. 1, according to an illustrative embodiment;

FIG. 2B is a schematic diagram of a drill bit in position to begin penetrating a target surface while a range finder measures the distance between the range finder and the target surface, according to an illustrative embodiment;

FIG. 2C is a schematic diagram of a drill bit penetrating a target surface while a range finder measures the distance between the range finder and the target surface, according to an illustrative embodiment;

FIG. 3A is a schematic diagram showing a pivoting range finder, according to an illustrative embodiment;

FIG. 3B is a schematic diagram showing a pivoting mirror for a range finding laser beam, according to an illustrative embodiment;

FIG. 4A is a schematic view of a laser range finder calibration system, according to an illustrative embodiment;

FIG. 4B is a schematic view of a laser range finder measuring a distance to a focus spot between a target surface and a drill bit tip, according to an illustrative embodiment;

FIG. 4C is a schematic view of a laser range finder measuring a distance to the focus spot after the drill bit has begun to penetrate the target surface, according to an illustrative embodiment;

FIG. 5A is a schematic view of a bone showing outer cortex and less dense interior, according to an illustrative embodiment;

FIG. 5B is a graph showing the speed of penetration and/or force required as the drill bit penetrates the different areas of the bone, according to an illustrative embodiment;

FIG. 6A is a schematic view of one possible implementation of an annular load cell within a depth sensing drill bit chuck and showing the annular load cell from the side, according to an illustrative embodiment;

FIG. 6B is a top view of the annular load cell of FIG. 6A, according to the illustrative embodiment;

FIG. 6C is a side view of a drill bit with an electrically conductive tip, according to an illustrative embodiment;

FIG. 6D is a cross section of the drill bit of FIG. 6C, taken along cross section line 6D-6D, according to an illustrative embodiment;

FIG. 6E is a cross section of the drill bit of FIG. 6C, taken along cross section line 6E-6E, showing a flex sensor, according to an illustrative embodiment;

FIG. 7 is a diagram showing a computing environment system for monitoring hole depth and determining ideal screw length, according to an illustrative embodiment; and

FIG. 8 shows a method of determining hole depth, according to an illustrative embodiment.

### DETAILED DESCRIPTION

There are a great many possible implementations of the invention, too many to describe herein. Some possible implementations are described below. In the following description, for purposes of explanation, numerous specific details are set forth in order to provide a thorough understanding thereof. It should be clear, however, that the innovation can be practiced without various specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, any particular embodiment need not have all the aspects or advantages described herein. Thus, in various embodiments, any of the features described herein from different embodiments may be combined. It cannot be emphasized too strongly, however, that these are descriptions of implementations of the invention, and not descriptions of the invention, which is not limited to the detailed implementations described in this section but is described in broader terms in the claims. Further, the terms and phrases used herein are not intended to be limiting but rather to provide an understandable description of the invention.

In various cases, a hole for a non-locking screw will typically extend the entire way through the bone from one side to the other. However, a locking screw can, but does not need to go all the way through the bone from one side to the other. Similarly, a hole for a locking screw does not need to extend the entire way through the bone from one side to the other. In the interest of simplicity, the description herein largely describes the use of non-locking screws and holes that extend entirely through the bone, however, it should be clear that in various embodiments, the description provided herein can apply to holes that do not need to extend entirely through the bone. It should also be clear that in various embodiments, the description provided herein can apply to various retention posts including locking screws, non-locking screws, pegs, rivets, bolts, or other fastening arrangements. As used herein, the term “screw” can refer to locking screws, non-locking screws, pegs, rivets, bolts, or other retention posts.

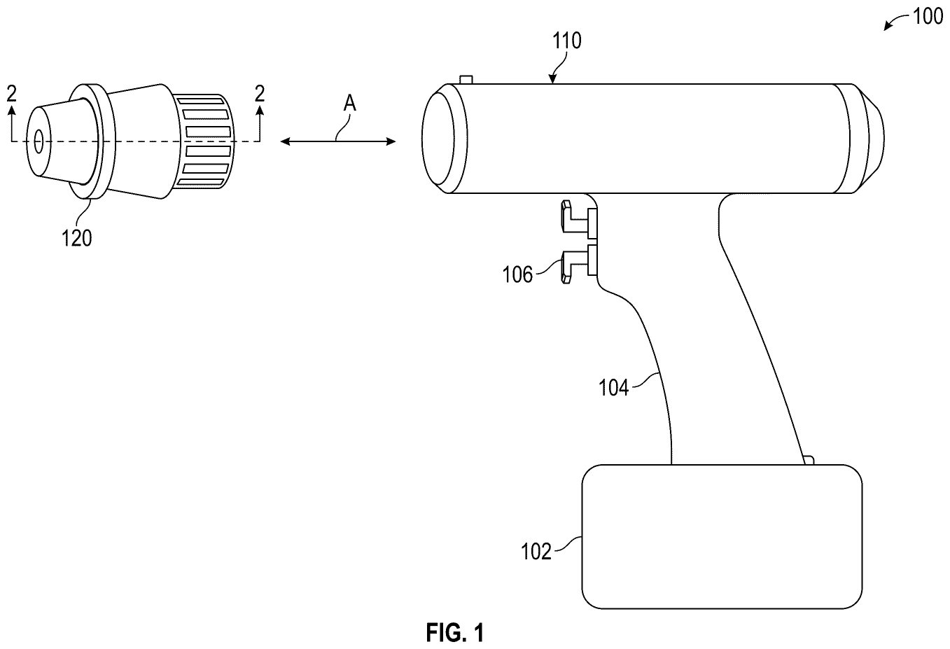

In various embodiments, a depth sensing drill system can include a depth sensing chuck that can determine the depth that a drill bit has been inserted below a surface. FIG. 1 is a perspective view of a depth sensing drill system, according to an illustrative embodiment. In various embodiments, a depth sensing drill system 100 can include a depth sensing chuck 120 that can be affixed to a standard handheld drill base 110. As shown and described herein, the handheld drill base 110 can be specialized medical equipment, such as a bone drill. The handheld drill base 110 can have a power source 102, a handle 104, and controls 106 that allow a user, such as a surgeon, to control the speed and direction of the drill's rotation. As used herein, the terms “surgeon” and “user” may be used interchangeably, however, it should be clear that the user does not need to be a surgeon, and anyone using the systems and methods described herein can be a user.

It should be clear that the depth sensing system described herein can be used in a variety of settings, and could be used with typical hardware type drills such as the types that may be found in most households, along with milling systems and others. It should also be clear that the depth sensing system described herein can be used to quickly, easily, and accurately determine various parameters of a drilled hole, including calculating the volume of a hole and the volume of material that has been removed to create the hole. It should further be clear that in various embodiments the depth sensing drill system described herein can include multiple units such as the depth sensing chuck 120 plus the handheld drill base 110, or the depth sensing drill system 100 can be integrated as a single unit.

In embodiments intended for use in hospitals and medical settings, a depth sensing chuck 120 can be a separate unit that can be removable from the reusable handheld drill base 110. The chuck 120 can be inserted and removed from the drill base in the direction shown by arrow A. The depth sensing chuck 120 can be removable, and can be sterilizable or disposable and replaceable. For simplicity purposes, the depth sensing chuck and drill base shown herein are a standard-type hospital quick connect system, however, it should be clear that the system described herein can be used in drill systems with various types of quick connect/disconnects, or in single-unit drill systems.

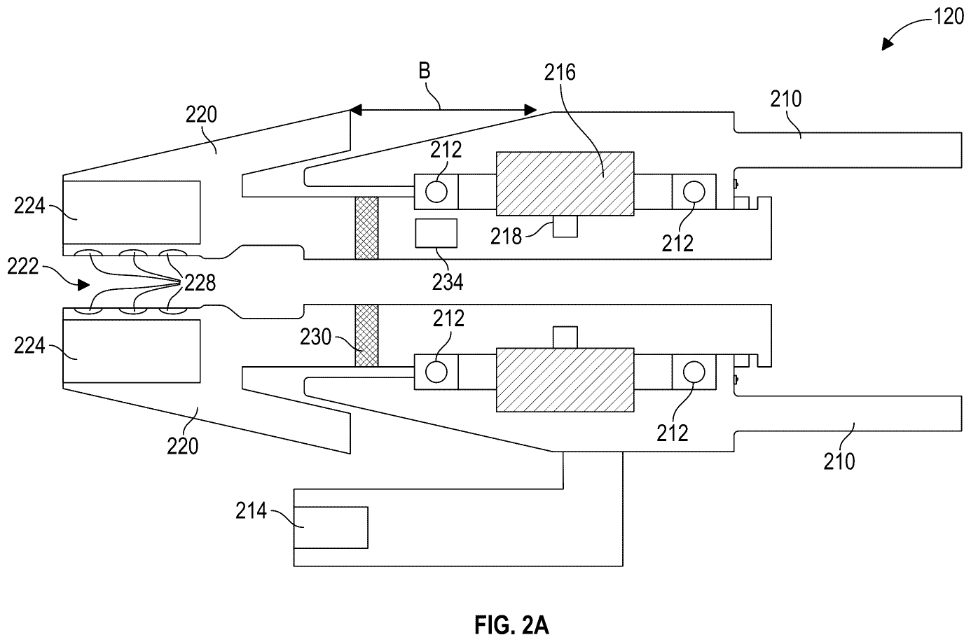

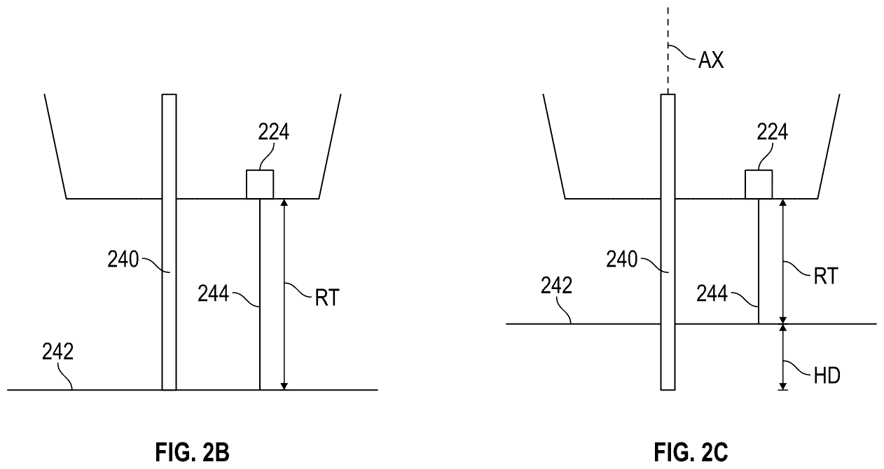

FIG. 2A is a cross sectional view of a depth sensing chuck, taken along cross section line 2-2 of FIG. 1, according to an illustrative embodiment. The chuck 120 can include a stationary portion 210 that can be engaged with the body of the drill base. In this way, the stationary portion 210 can remain in a fixed position relative to the drill base body, the handle and the controls. The depth sensing chuck 120 can also have a rotary portion 220. Rotary portion 220 can be operatively engaged with the motor of the drill base, so that the motor of the drill base rotates the rotary portion 220. Rotary portion 220 can also hold a drill bit within the bit clamp area 222. In this way, the motor of the drill base can spin the drill bit that is held within the bit clamp area 222.

Various intermediary components such as washer bearings 212 can allow the rotary portion to spin 220 relative to the stationary portion 210. A user can urge the rotary portion 220 towards the stationary portion 210 along the direction shown by arrow B to open the drill clamp area 222 thereby allowing a drill bit or other tool to be inserted or removed, and a user can urge the rotary portion away from the stationary portion along arrow B, thereby causing the drill clamp to close around the drill bit and hold the bit secure and ready for drilling. It should be clear that the specific designs, clamping mechanisms, bearings, etc. can all be variable between different designs, however, it should be clear to one skilled in the art how various designs for rotating drill bit clamps can operate on similar principles.

A range finder can bounce various forms of energy waves off of a surface and determine the distance from the range finder to the measured surface. In various embodiments, a range finder can use energy waves such as electromagnetic radiation, sonic waves, ultrasonic waves, or various other forms of energy waves that can be used to measure distance. As described herein, a depth sensing chuck can use a laser range finder to measure the distance from the chuck to a target surface, however, various range finders that utilize various electromagnetic radiations, sonic waves, ultrasonic waves, or others are possible. A depth sensing chuck 120 can have one or more range finders that can include rotary laser range finders 224 and/or stationary laser range finders 214. As the drill bit penetrates the target surface, such as a bone or other surfaces, the laser range finder can measure how far the drill has penetrated the surface by measuring the decreasing distance between the range finder and the target surface.

In various embodiments, a depth sensing chuck 120 can include a dynamo 216 that can be situated between the rotary portion 220 and the stationary portion 210. Dynamo 216 can generate electrical energy from the relative rotary motion between the rotary portion and the stationary portion. In this way, dynamo 216 can provide electrical energy to various electric components such as the one or more range finders, along with any communication equipment, load sensors, and/or other electronic components of the depth sensing drill bit chuck, described in more detail below. In various embodiments, the depth sensing drill chuck can also include a small battery 218 adapted to store energy generated by the dynamo, so that the supply of electrical energy to the various electric components is not disrupted when the drill stops spinning. As used herein, the term “power source” can be used to refer to the dynamo 216, the battery 218, or both.

FIG. 2B is a schematic diagram of a drill bit 240 in position to begin penetrating a target surface 242 while a range finder 224 uses a laser beam 244 to measure the distance between the range finder and the target surface, according to an illustrative embodiment, and FIG. 2C is a schematic diagram of a drill bit 240 penetrating a target surface 242 while a range finder 224 uses a laser beam 244 to measure the distance between the range finder and the target surface, according to an illustrative embodiment. In various applications of the system described herein, a target surface can be flat and the drill bit can be inserted perpendicular to the flat target surface. In that most simple application, a depth sensing chuck with a single and/or stationary range finder 214 can easily determine drill bit penetration distance by measuring the range finger-to-target distance RT, from the range finder to the target surface, as the drill bit penetrates the target surface. As the distance RT decreases, the drill pit penetration distance increases. Drill bit penetration distance can also be referred to as hole depth HD, and the change in distance between the range finder and the target surface can be calculated to find the hole depth. Drill bit length TDL remains constant during drilling, while measured distance RT decreases as hole depth HD increases.

However, in some settings, the situation may be more complicated. The target surface, such as the exterior of a bone, can be curved and can include concave and convex areas. Furthermore, the angle of penetration may not be perpendicular to the target surface. In order to overcome this, in various embodiments a laser range finder 224 itself can rotate around the central axis. As the laser rotates around the central axis AX of the drill bit, the measured distance RT can change at different rotary positions around the circle due to the uneven target surface. In various embodiments, the measured distance RT can be measured at multiple rotary positions as the laser travels around the 360-degree rotary path, and the measured distances can be averaged to calculate the average measured distance, and the averaged measured distance can be used to determine the drill bit penetration distance. By averaging measured distances collected from multiple rotary positions, the depth sensing drill chuck can increase the accuracy of the penetration depth determination, even as the drill bit is penetrating curved or uneven surfaces, and even as the drill bit is penetrating surfaces at angles that can be different from 90 degrees. Without averaging distances collected from multiple rotary positions, the measured distance may fluctuate depending on various factors such as whether the surgeon allows the drill body to move to various rotary positions around the drill bit axis during drilling.

In various embodiments, measurements can be recorded at the same rotary position as the rotary portion rotates. As a possible alternative to averaging, or in combination with averaging, measurements can be recorded for one or more specific rotary positions as the rotary portion rotates. By measuring at the same rotatory position for each measurement, the travel distance, and hole depth HD, can be easily calculated as the measured distance RT decreases, similar to measurements taken with a non-rotating range finder.

In various embodiments, a processor 234 can perform averaging, can calculate hole depth HD, and can perform various other calculations that will be described more fully below. In various embodiments, one or more processors 234 can be within the chuck, can be within the drill body, and/or can be outside of the drill.

In various embodiments, a depth sensing drill bit chuck can have more than one range finder, and multiple lasers may be at different radial distances from the central axis, and/or multiple lasers may be at the same radial distance from the central axis. Multiple simultaneous measurements from different and/or same radial distances can increase overall measurement accuracy throughout the process, including at the moment of penetration, as discussed further below. In various embodiments, lasers may rotate at the same speed or different speed as the drill bit chuck. In various embodiments, the lasers may rotate while the drill bit remains stationary. In various embodiments, laser rotation and rotation speeds may be independently controlled by the user.

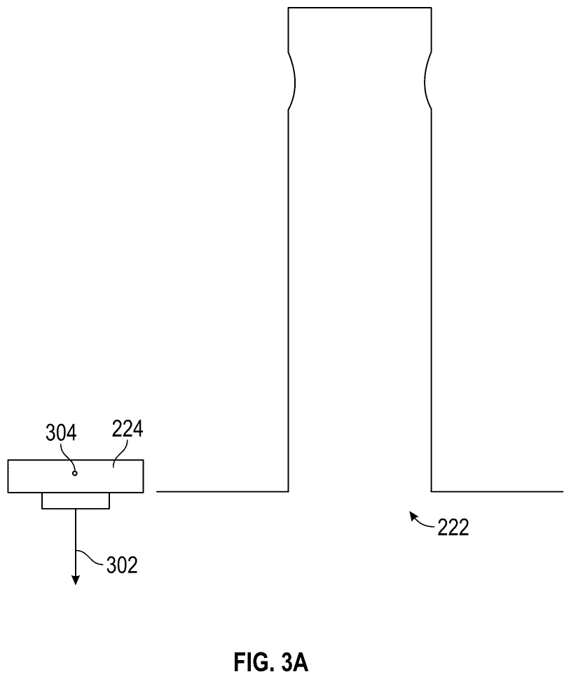

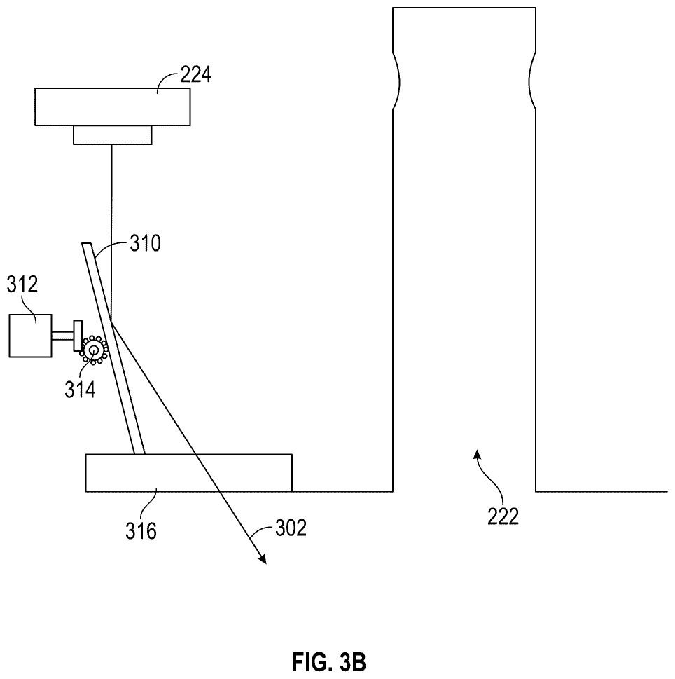

In various embodiments a laser range finder itself can pivot, or a laser beam can be directed off of a mirror that can pivot. FIG. 3A is a schematic diagram showing a pivoting range finder, according to an illustrative embodiment. A pivoting range finder can use a servo or other motor to aim a laser beam. The laser range finder 224 can pivot around axis 304 so that the aimable laser beam 302 can be directed towards the drill bit in a range of angles. FIG. 3B is a schematic diagram showing a pivoting mirror for a range finding laser beam, according to an illustrative embodiment. The laser range finder 224 can direct an aimable laser beam 302 towards a mirror 310. The mirror can be driven by motor to pivot around pivot point 314, so that the mirror can direct the aimable laser beam towards the drill bit in a range of angles as the mirror pivots around pivot point 314. Aimable laser beam 302 can pass through a window 316 that can be made of glass, plastic, or other light-permeable and sterilizable materials. It should be clear to a person of skill in the art that stationary range finder 214 and/or a rotary range finder 224 can have pivots 304 and/or pivoting mirrors 310 so that they can direct an aimable laser beam 302 at a point of focus at a junction of the drill bit and the laser beam. An aimable laser beam can be aimed using movement of the laser emitter, rotation of a prism, rotation of a mirror, or various other means that would be known to one skilled in the art, so that the point of focus of the laser can be changed as the drill advances.

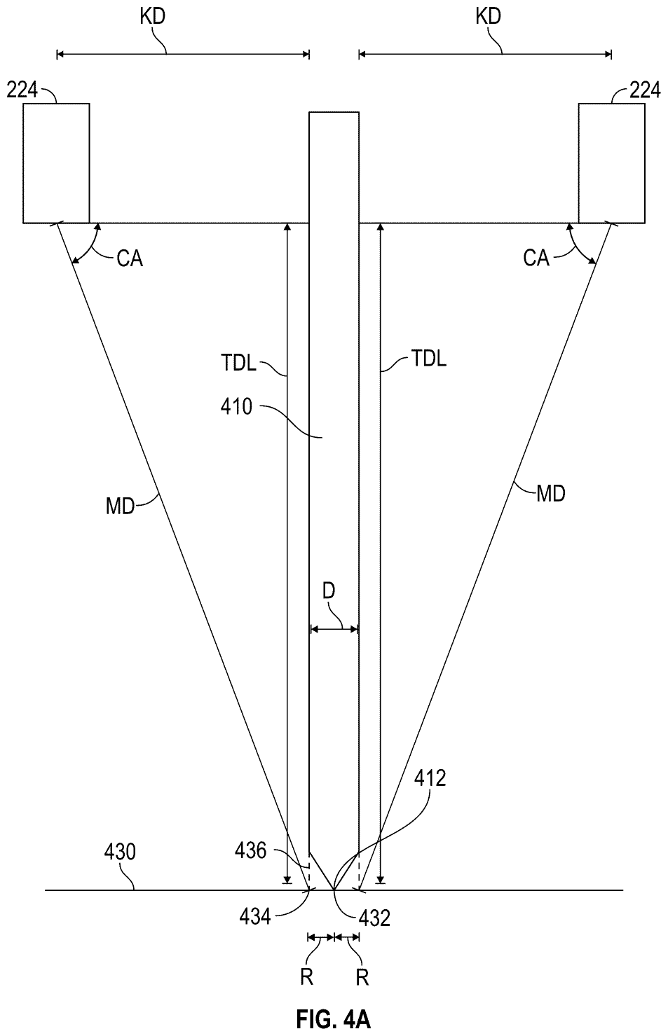

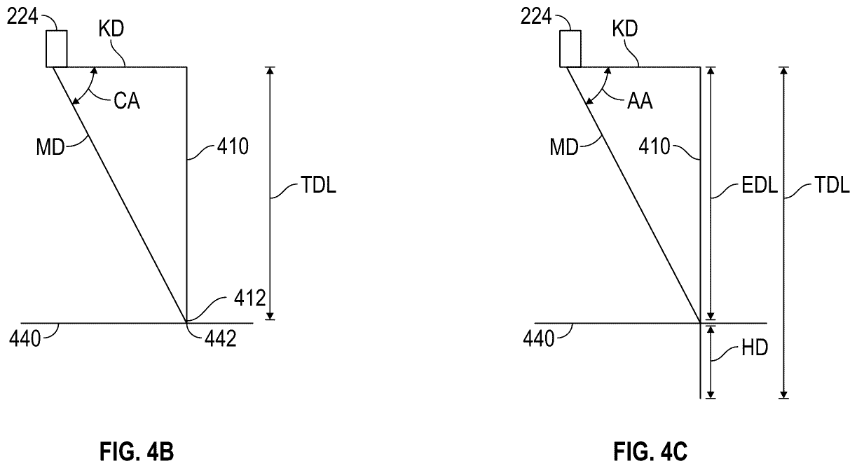

FIG. 4A is a schematic view of a laser range finder calibration system, according to an illustrative embodiment. Before the drill bit 410 is used to drill into a bone or other target surface, the laser range finder 224 can be calibrated. The drill bit tip 412 can rest on the calibration surface 430, and the aimable laser beams from the laser range finders 224 can be brought to aim at the area where the drill bit tip 412 contacts the calibration surface 430.

Contact spot 432 can be the exact spot where the drill bit tip 412 contacts the calibration surface, D can be the diameter of the drill bit, and R can be the length of a radius from the central axis of the drill bit to the outer surface of the drill bit, with D=2R. A ring on the calibration surface can have a center at the contact point 432 and a radius R. The aimable laser beam can be aimed at a focus spot 434 that can be a spot on the ring that is nearest to the rotary range finder 224, or put another way, the aimable laser beam can be aimed at a focus spot 434 where the outer surface of the drill bit would contact the calibration surface if the calibration surface was penetrated by the drill bit to a depth that allows the full diameter of the drill bit to contact the calibration surface. The focus spot 434 can be on a line 436 that extends along the outer surface of the drill bit shaft and is parallel to the central axis of the drill bit. In various embodiments, the focus point 434 can simply be the contact spot 432 itself.

As explained above, the focus spot 434 can be anywhere on an imaginary circle where the side of the drill bit meets the bone. The focus spot can be a spot anywhere around that ring, and the focus spot can move around the ring as the rotary range finder rotates around. In various embodiments, the focus spot 434 can be radially distant from the side of the drill bit, so that, for example, the focus spot is on a ring that can be larger than the diameter D of the drill bit. That is to say, the focus spot can be on a concentric circle that is slightly larger than the diameter of the drill bit, extending radially outwards from the circle where the side of the drill bit meets the bone. By way of non-limiting example, the focus spot can be approximately 1-2 mm out from the side of the drill bit.

The aimable laser beam can be aimed at the focus spot 434 at calibration angle CA. Using the measured distance MD between the range finder 224 and the focus spot 434, and the known distance KD, the Pythagorean theorem can provide the total drill bit length TDL. In various embodiments the calibration surface can be a specialized surface, and in various embodiments the calibration surface may have a ring on the calibration surface that corresponds to the diameter D of the drill bit. In various embodiments, the bone of the patient can be used as a calibration surface, and the user can calibrate while the drill bit is in contact with the patient, moments before the user begins drilling.

FIG. 4B is a schematic view of a laser range finder measuring a distance to a focus point between a target surface and a drill bit tip, according to an illustrative embodiment. As shown in FIG. 4B, the drill bit 410 has not yet begun to penetrate the target surface 440, and the total drill bit length TDL is outside of the bone. The total drill bit length TDL is known from the calibration step performed earlier, as shown in FIG. 4A, and the laser beam is aimed at the focus spot 442 at calibration angle CA.

FIG. 4C is a schematic view of a laser range finder measuring a distance to the focus spot after the drill bit has begun to penetrate the target surface, according to an illustrative embodiment. As the drill bit 410 penetrates the target surface 440, the laser range finder 224 can detect the decrease in distance between the range finder and the target surface, and the angle AA of the aimable laser 302 can be adjusted so that the laser continues to point at the focus spot 442. As the measured distance MD decreases, a processor can calculate a new angle AA in order to keep the laser aimed at the same focus spot. In various embodiments, one or more laser range finders can include one or more cameras, and the cameras can provide information about the laser aiming, so that the angle AA can be adjusted to keep the laser pointed at the focus spot. The portion of the total drill bit length TDL that remains exposed is the exposed drill bit length EDL, and the exposed drill bit length EDL can be calculated using the known distance KD and the measured distance MD.

As the drill bit 410 penetrates the target surface 440, the portion of the total drill bit length TDL under the target surface 440 represents the increasing hole depth HD. The exposed drill bit length EDL and the hole depth HD added together equal the total drill bit length TDL. Therefore, the hole depth HD can be calculated by subtracting the exposed drill bit length EDL that can be determined in real time while drilling from the total drill bit length TDL that was determined during the calibration phase.

Drilling a hole at an acute angle to the target surface can be particularly difficult for a surgeon, and it was especially difficult to measure hole depth using a traditional depth gauge on an acute-angled hole. However, the use of two or more laser beams together, as shown in FIG. 2A, can help to improve accuracy of depth measurements in real time, at nearly any angle. Having laser beams on opposite sides of the chuck allows for measurements from both sides of the contract point while the surgeon is still drilling. These two measurements can be averaged to reach a more accurate distance number. Similarly, mounting a laser on the rotary portion allows for measurements to be taken from all positions around the drill bit, resulting in higher accuracy. In various embodiments, a depth sensing chuck can have two or more lasers, and the two or more lasers can be mounted on the rotary portion to increase accuracy. In this way, the depth sensing bone drill can determine the hole depth in real time as the surgeon is pushing the drill into the bone.

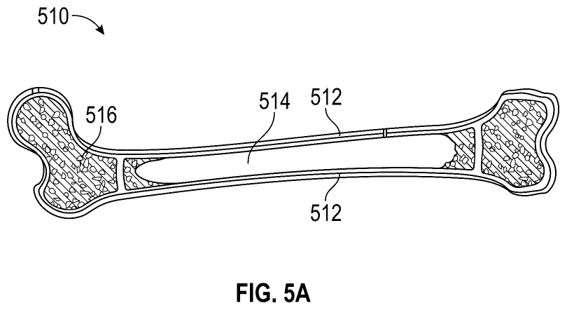

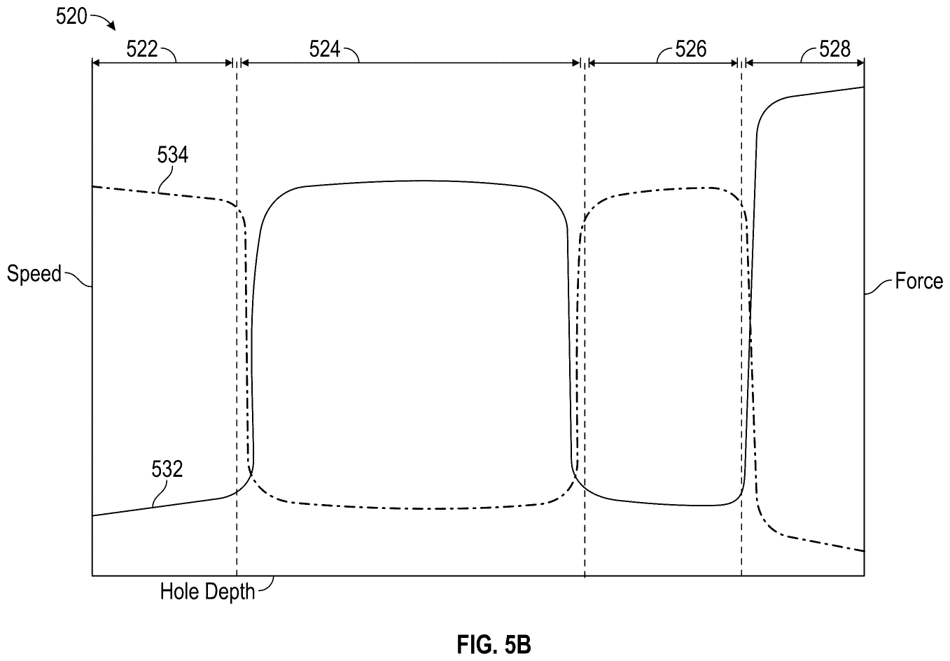

Most bones do not have uniform density. FIG. 5A is a schematic view of a bone showing the hard outer cortex and the less dense interior medulla, according to an illustrative embodiment. Bones 510 typically have a dense outer cortex 512, and a somewhat less dense interior medulla 514 and somewhat less dense cancellous area 516. In a typical bone, the medulla 514 and cancellous area 516 are both noticeably less dense than the more dense exterior outer cortex 512.

As the surgeon pushes the drill bit through the outer cortex 512, the pushing force required is greater and/or the penetration speed is lower. Then after breaking through the cortex, the drill bit tip is passing through the less dense interior medulla 514 and or cancellous area 516, and the drill bit passes through the bone more easily. The drill bit tip can penetrate the interior medulla 514 and or cancellous area 516 more quickly and/or penetrate with less force required from the surgeon. Finally, as the drill bit tip passes through to the other side of the bone and reaches the far cortex again, the pushing force is higher and/or the speed of penetration is lower while passing through the cortex compared to the less dense interior medulla and/or cancellous area. The medulla 514 and cancellous area 516 are both substantially less dense than the outer cortex 512, and as used herein, the terms medulla 514 and cancellous area 516 can be used interchangeably to describe the interior of the bone that is significantly less dense than the outer cortex 512.

As the depth sensing chuck system senses the drill bit depth, also referred to as the penetration depth, the system can also monitor how the depth changes over time. Put another way, the system can determine the speed at which the drill is penetrating the bone. FIG. 5B is a graph 520 showing the speed of penetration and/or force required as the drill bit penetrates the different areas of the bone, according to an illustrative embodiment. The X axis indicates the distance traveled by the drill bit tip, which is the Hole Depth HD. As the drill bit tip travels, it passes through different zones with different densities. Zone 522 is indicative of the drill bit tip passing through the cortex 512 the first time. Zone 524 is indicative of the drill bit tip passing through the less dense interior medulla 514. Zone 526 is indicative of the drill bit tip passing through the outer cortex 512 on the other side, and zone 528 is indicative of the drill bit tip having exited out the from the far side of the cortex into soft tissue.

The left-side Y-axis indicates speed, and graph line 532 shows the speed of penetration as the drill bit tip passes through each zone. As shown, the speed of penetration is slowest as the drill bit passes through the cortex zone 522, faster as the drill bit tip passes through the less dense interior medulla zone 524, then slowest again as the drill bit passes through the cortex zone 526 on the other side. As the drill bit tip exits the cortex zone 526 on the far side of the bone, the speed can increase significantly in zone 528 because the surrounding soft tissue provides minimal resistance to the drill bit. By monitoring the speed of penetration, the depth sensing drill chuck can determine the exact moment the drill bit tip passes out of the cortex and into the surrounding soft tissue.

Because the depth sensing drill bit chuck is also monitoring the hole depth at the same time as monitoring speed, the depth sensing drill bit chuck can quickly determine the hole depth at the moment the drill bit tip crosses from the second cortex zone 526 into the soft tissue zone 528. The drill bit depth at the moment the speed increases from zone 526 to zone 528 can be the correct depth of the hole and therefore the best screw length. In this way, the surgeon does not need to attempt to measure the hole depth using a depth gauge, because the depth sensing chuck has already determined the hole depth at the moment the drill bit tip passes out of the cortex. The depth sensing chuck is constantly determining the hole depth in real time as the hole is being drilled, and can the provide the exact depth when the drill bit has completed the hole through the bone.

The right-side Y-axis indicates the force used by the surgeon to push the drill bit tip through the various zones, and graph line 534 shows the force used as the drill bit tip passes through each zone. As shown, the force used is highest as the drill bit passes through the more dense cortex zone 522, lower as the drill bit tip passes through the less dense interior medulla zone 524, then highest again as the drill bit passes through the cortex zone 526 on the other side. As the drill bit tip exits the cortex zone 526 on the far side of the bone, the force required drops off significantly in zone 528 because the surrounding soft tissue provides minimal resistance to the drill bit. By monitoring the force applied to the drill by the surgeon, the depth sensing drill chuck can determine the exact moment the drill bit tip passes out of the cortex and into the surrounding soft tissue.

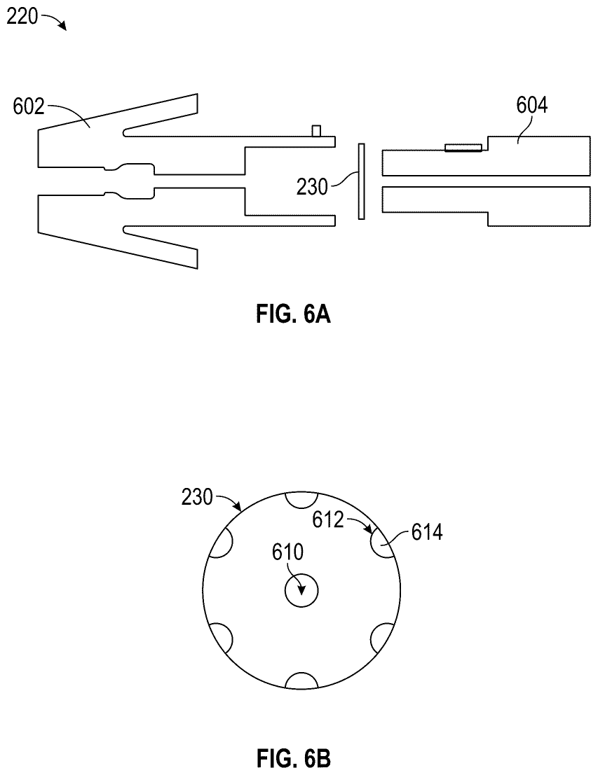

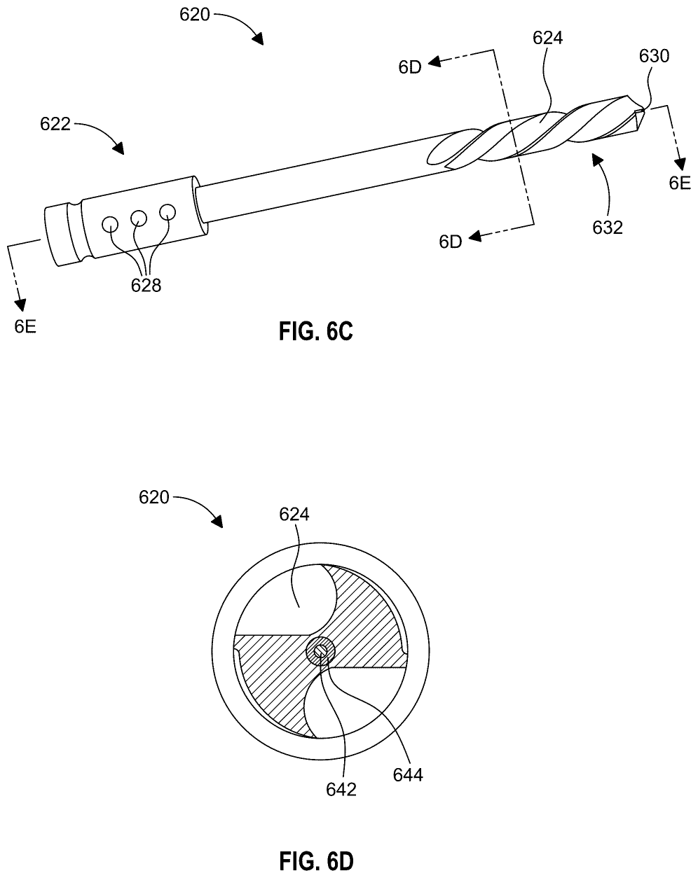

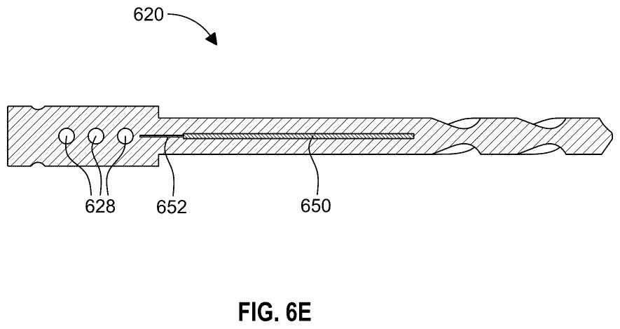

Turning briefly to FIG. 2A, a force sensor 230 such as an annular load cell is shown within rotary portion 220, however, it should be clear that in various embodiments, various load cells 230 can be placed in various locations to measure the force applied by the surgeon to the drill bit tip. FIG. 6A is a schematic view of one possible implementation of an annular load cell within a depth sensing drill bit chuck and showing the annular load cell from the side, according to an illustrative embodiment, and FIG. 6B is a top view of the annular load cell of FIG. 6A, according to the illustrative embodiment. The rotary portion 220 can have a front portion 602 and a back portion 604. The annular load cell 230 can be seated within a pocket between the front portion 602 and the back portion 604. The annular load cell can have a central opening 610 that can allow the drill bit to be seated within the rotary portion and/or can allow a guide wire to pass through the central opening. In various embodiments, the annular load cell can include one or more notches 612, and the rotary portion 220 can have various corresponding pegs or other protuberances 614 that engage with the notches 612 to hold the load cell 230 in a fixed location and fixed position.

Turning now to FIGS. 2A, 5B, and 6A, because the depth sensing drill bit chuck is also monitoring the hole depth at the same time as monitoring applied force, the depth sensing drill bit chuck can quickly determine the hole depth at the moment the drill bit tip crosses from the second cortex zone 526 into the soft tissue zone 528. The drill bit depth at the moment the force decreases from zone 526 to zone 528 can be the correct depth of the hole and therefore the best screw length. In this way, the surgeon does not need to attempt measure the hole depth using a depth gauge, because the depth sensing chuck has already determined the hole depth at the moment the drill bit tip passes out of the cortex.

In various embodiments, a drill bit can include electrical connectivity at or near the tip of the drill bit. FIG. 6C is a side view of a drill bit with an electrically conductive tip, according to an illustrative embodiment. The drill bit 620 can have a tip region 632, drill flutes 624, and a base region 622 that engages with the bit clamp area of the chuck. The electrically sensitive drill bit 620 can have one or more electrical sensors 630 in the tip region 632. In various embodiments, the one or more electrical sensors 630 can be an exposed end of a conductive wire, filament, or other electrically conductive channel that can run through or along the drill bit. In various embodiments, the one or more electrical sensors 630 can be electrodes or other sensors that can be electrically connected to the conductive wire, filament, or other electrically conductive channel. The wire, filament, or other electrically conductive channel can electrically connect the sensor(s) 630 in the tip region 632 to electrical connectors 628 near the base of the drill bit. Electrical connectors 628 can connect to corresponding electrical connectors 228 that can be on the inside of the bit clamp area 222.

Turning briefly to FIG. 2A, a bit clamp area 222 of a depth sensing chuck 120 can have electrical connectors 228. Electrical connectors 228 can operatively connect the electrical connectors 628 of a drill bit to a power source, a processor 234, communication systems such as Bluetooth or others, and/or other possible electrical connections. Connectors 228 can be arranged in a pattern that corresponds to connectors 628 on a drill bit. The electrical sensors 630 of the drill bit can thereby be operatively connected to a power source, a processor, communications, and/or other possible electrical connections. Connectors 228 and/or 628 can be annular connectors that can make the connection at any rotational position of the drill bit 620 within the chuck 120. Connectors 228 and 628 can be held together by the pressure of the chuck holding the drill bit in place.

Turning now to FIGS. 2A and 6C, an electrically sensitive drill bit 620 can have one or more electrical sensors 630 in the tip region 632, and the electrical sensors can be connected to a power source, a processor, communication, and/or other possible electrical connections. In various embodiments, the electrically connective channels can be insulated to isolate the electrically connective channels away from the rest of the drill bit.

The one or more electrical sensors 630 can send data to a processor that can be in the chuck, the drill base, or other locations. Various information can be transmitted to the processor, including the electrical conductivity of the material being drilled. In various embodiments, the drill bit may have two or more electrical sensors 630 that can be electrodes near the tip region of the drill bit. In various embodiments, the outside of the drill bit may be one of two or more electrodes, with an additional electrode being an electrical sensor 630 near the tip of the drill bit and insulated from the rest of the drill bit. An electrical charge can be sent to the electrode(s), and the conductivity of the material being drilled can be determined by the processor. Various other sensors are also possible, and the sensed data about the material being drilled can be transmitted to the processor.

As the drill bit passes through various layers of material, sensors such as electrical sensors 630 can measure various aspects of the material being drilled. As the drill bit passes from one layer of material to another, the sensed aspects of the drilled material, such as electrical conductivity, can change from layer to layer. As the processor senses the changes in various layers, the processor can correlate that data with the hole depth at that moment, so that the aspects of the material being drilled can be known at each hole depth. The depths at which the hole passes from one material into another can be known with precision, so that the user can know the exact hole depth at each point where the drill bit passes into a different material.

FIG. 6D is a cross section of the drill bit of FIG. 6C, taken along cross section line 6D-6D, according to an illustrative embodiment. In various embodiments, one or more insulated wires 642 can pass through the drill bit. Insulated wires 642 can be insulated from the drill bit by insulation 644 that can be various non-conductive materials. Insulated wires 642 can link the one or more sensors 630 to the connectors 628. In various embodiments, insulated wires 642 can transfer electric signals and/or other data through the drill bit without sending current or power through the rest of the drill bit.

FIG. 6E is a cross section of the drill bit of FIG. 6C, taken along cross section line 6E-6E, showing a flex sensor, according to an illustrative embodiment. Drill bit 620 can have a flex sensor 650 within the drill bit. Non-axial forces acting on the drill bit may cause the drill bit to flex slightly. Flex sensor 650 can detect non-axial forces acting on the drill bit, so that the user and/or processor can be aware of any forces that are not directly pressing along the central axis of the drill bit. Any information about non-axial forces can be transmitted from the flex sensor 650 along a data wire 652, which can pass through connectors 628 and 228 to the processor. This system can provide sensitivity to skiving or deflecting of the drill bit off of hard surfaces, such as a surgical bit deflecting off of a cortical bone. This can also be useful, for example, when drilling pedicle screws during spine surgery. The system described herein can help a surgeon to avoid incorrect penetration of a vertebrae cortex by providing information about skiving or deflection, or other information about non-axial forces. Information about non-axial forces can be useful to a user who may not be aware that the drill bit is experiencing non-axial forces.

Turning now to FIGS. 6C-6E, the one or more sensors 630 can be located anywhere on the drill bit, including at or near the tip, in or around the flutes, within the interior of the drill bit, and/or along the side of the drill bit. A processor can receive the sensed information, which may include electrical conductivity, from the sensor(s). In various embodiments, the processor may receive information in the form of current and/or impedance from the electrical signal passing through the material being drilled. The processor can then use the information on changing conductivity between layers combined with instantaneous depth measurements to determine the hole depths where the drilled material changes. Changes in drilled material can include penetrating from the cortex into the interior medulla, penetrating from the interior medulla to the cortex, or penetrating through the far side of the bone and into soft tissue.

In various embodiments, a processor can detect changes in electrical connectivity to determine cortical penetration, and the processor can provide a hole depth at the moment of cortical penetration as an output. In various embodiments, a processor can detect changes in acceleration to determine cortical penetration, and the processor can provide the hole depth at the moment of cortical penetration as an output. In various embodiments, a processor can detect changes in applied force between the drill body and the drill bit to determine cortical penetration, and the processor can provide the hole depth at the moment of cortical penetration as an output. In various embodiments, a processor can detect changes in acceleration, changes in applied force, changes in electrical conductivity, and/or various other changes to determine cortical penetration, and the processor can provide the hole depth at cortical penetration as an output. It should be clear that similar systems and methods can be applied to other forms of drilling, milling, etc., such as by sensing the moment the drill bit tip passes through the other side of the drilled object.

In various embodiments, similar systems can be used in fracking, mining, construction, plumbing, or various other applications. Various sensors can be configured to measure conductivity, humidity, temperature, hardness, gas, or any materials, minerals, metals, etc., including various materials such as wool, precious metals such as gold, valuable materials such as oil, or others. By way of non-limiting examples, similar systems may detect different underground layers for use in mining and/or fracking. Similar systems may detect when a drill bit has passed through sheetrock, or when a drill bit has hit a stud, a wire, a plumbing pipe, or other materials. In various embodiments, a processor may be able to map out different material layers at different hole depths in a wide range of industries and applications.

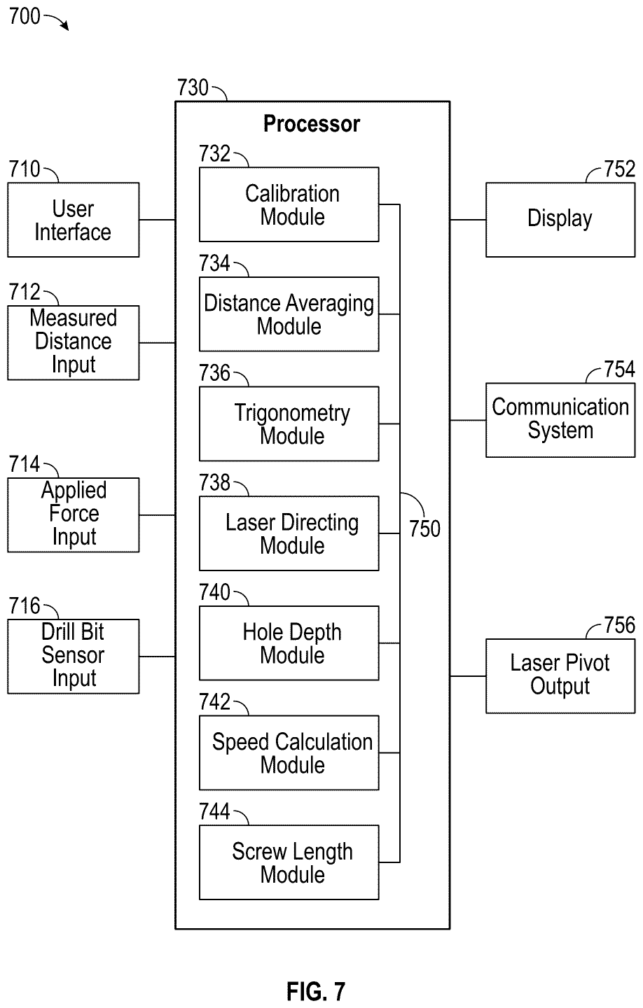

FIG. 7 is a diagram showing a computing environment system for monitoring hole depth and determining ideal screw length, according to an illustrative embodiment. In various embodiments, a system 700 for monitoring hole depth and determining ideal screw length can include a processor 730 and a user interface 710. The range finder can determine measured distance, and the processing system can have a measured distance input 712. Measured distance input 712 can provide the measured distance to the processor 730. The load cell can determine the applied force, and the processing system can have an applied force input 714. Applied force input 714 can provide the measured applied force to the processor 730. In various embodiments, a system 700 can have a drill bit sensor input 716. Drill bit sensor input 716 can provide the sensed data such as conductivity to the processor 730.

The processor can have a calibration module 732 that can use trigonometry to calibrate and determine the total drill bit length before drilling begins. The processor can have a measured distance averaging module 734. In embodiments with one or more rotating range finders, the measured distance averaging module 734 can average the measured distances that can be measured at multiple rotary positions as a range finder travels around the 360-degree rotary path. The measured distance averaging module 734 can calculate an average measured distance from the distances measured by one or more rotating range finders. The averaged measured distance can then be used as the measured distance in further calculations. Averaging the measured distances from one or more range finders rotating around a 360 degree rotary path can increase the accuracy of the penetration depth calculation in real time, especially in cases where the drill bit is penetrating an uneven surface and/or penetrating a surface at an angle that is not a 90-degree angle.

The processor can have a trigonometry module 736 that can use the measured distance and the known distance to calculate the exposed drill bit depth as the drill bit is penetrating the surface. The processor can have a laser directing module 738. Laser directing module 738 can provide a laser pivot output 756 to the laser motor 312 providing commands for the laser motor to adjust the angle of the aimable laser. The laser directing module can provide commands to the laser motor to keep the aimable laser beam directed at the desired focus point in real time as the drill penetrates the bone. The processor can have a hole depth module 740 that can calculate hole distance from the total drill bit length and the exposed drill bit length. The processor can have a speed calculation module 742 that can determine speed of drill bit penetration from the change in hole depth over time. The processor can have a screw length determining module 744 that can use the change in force and/or the change in speed to determine the moment that the drill bit tip passes out of the second cortex layer, and determine the hole depth at that moment. The processor can have a bus 750 that can connect various modules.

The system can include a display 752 that can display the ideal screw length after drilling is complete. The display 752 can also display various information including real-time penetration depth, penetration speed, and/or applied force. The system can include a communication system 754. Communication system can include various wireless communication wavelengths, so that the depth sensing drill bit chuck can send and receive information from external systems. The communication system 754 can communicate with an external user interface and/or an external display.

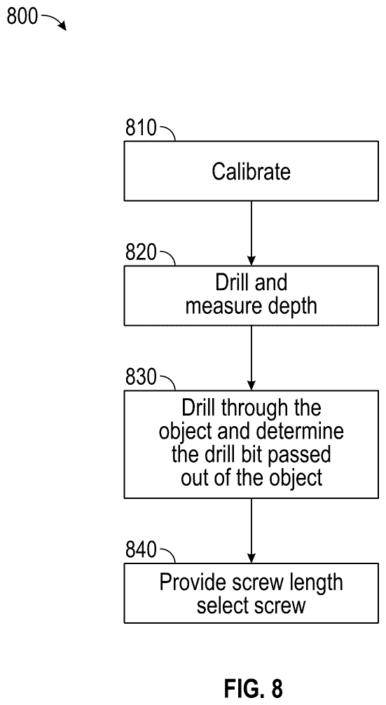

FIG. 8 shows a method of determining hole depth, according to an illustrative embodiment. A method of determining hole depth 800 includes calibrating the total drill bit length at box 810. At box 820, a user can drill into a target surface. As the user is drilling into the target surface, the depth sensing drill bit chuck can determine the hole depth in real time, as the drill bit is penetrating the target surface. At box 830, the user can drill through the target object, such as the bone, and the depth sensing drill bit chuck can determine that the drill bit tip has passed through the object and out through the other side. At box 840, the depth sensing drill bit chuck can provide the user with the ideal screw length, and the user can select a screw with the ideal screw length.

Although this invention has been disclosed in the context of certain preferred embodiments and examples, it therefore will be understood by those skilled in the art that the present invention extends beyond the specifically disclosed embodiments and/or uses of the invention and obvious modifications and equivalents thereof.

Thus, it is intended that the scope of the present invention herein disclosed should not be limited by the particular disclosed embodiments described above, but should be determined only by a fair reading of the claims that follow.

Some embodiments, illustrating its features, will now be discussed in detail. The words “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

References to “one embodiment”, “an embodiment”, “another embodiment”, “one example”, “an example”, “another example” and so on, indicate that the embodiment(s) or example(s) so described may include a particular feature, structure, characteristic, property, element, or limitation, but that not every embodiment or example necessarily includes that particular feature, structure, characteristic, property, element or limitation.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. Also, as used herein, various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 5%) of the system. Note also, as used herein the terms “process” and/or “processor” should be taken broadly to include a variety of electronic hardware and/or software based functions and components. Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Accordingly, this description is meant to be taken only by way of example, and not to otherwise limit the scope of this invention.

What is claimed is:

1. A depth sensing drill chuck comprising:
a drill bit securing area adapted to secure a drill bit;
at least one range finder adapted to measure a distance between the depth sensing drill chuck and a target surface; and
a processor that uses the measured distance between the depth sensing drill chuck and the target surface to calculate a penetration depth in real time as the target surface is being penetrated.

2. The depth sensing drill chuck of claim 1, wherein the depth sensing drill chuck comprises a stationary portion adapted to be engaged with a body of drill base and a rotary portion adapted to be operatively engaged with a motor of the drill base, the depth sensing drill chuck further comprising an electricity-generating dynamo between the stationary portion and the rotary portion.

3. The depth sensing drill chuck of claim 1, further comprising a battery adapted to maintain an uninterrupted power supply to the range finder and the processor after the rotary portion has stopped spinning.

4. The depth sensing drill chuck of claim 1, further comprising a load cell adapted to measure force applied to the drill bit as the drill bit penetrates the target surface, wherein the processor uses the measured distance and the measured force to calculate a penetration depth in real time as the target surface is being penetrated.

5. The depth sensing drill chuck of claim 1, further comprising a laser aiming motor that keeps the laser focused on a focus point on the target surface as the depth sensing drill chuck moves closer to the target surface during drilling.

6. The depth sensing drill chuck of claim 1, wherein the range finder is located on the rotary portion so that the distance between the depth sensing drill chuck and the target surface can be measured at multiple locations around the 360-degree rotary path.

7. The depth sensing drill chuck of claim 6, wherein the processor further comprises a measured distance averaging module that averages the measured distances at multiple rotary positions to calculate an averaged measured distance that can be used as the measured distance to calculate the penetration depth.

8. The depth sensing drill chuck of claim 1, wherein the processor further comprises a speed calculation module that monitors the change in penetration depth over time to determine a speed at which the target surface is being penetrated, and wherein the processor further comprises a screw length determining module adapted to use a change in speed to determine the moment that the drill bit tip passes out of a second cortex layer of a bone.

9. A depth sensing drill chuck comprising:
at least one range finder adapted to measure a distance between the depth sensing drill chuck and a target surface; and
a load cell adapted to measure force applied to a drill bit as the drill bit penetrates a target surface.

10. The depth sensing drill chuck of claim 9, wherein the depth sensing drill chuck comprises a stationary portion adapted to be engaged with a body of drill base and a rotary portion adapted to be operatively engaged with a motor of the drill base, the depth sensing drill chuck further comprising an electricity-generating dynamo between the stationary portion and the rotary portion.

11. The depth sensing drill chuck of claim 10, further comprising a battery adapted to maintain an uninterrupted power supply to the range finder and the load cell after the rotary portion has stopped spinning.

12. The depth sensing drill chuck of claim 9, further comprising a laser aiming motor that keeps the at least one laser focused on a focus spot on the target surface as the depth sensing drill chuck moves closer to the target surface during drilling.

13. The depth sensing drill chuck of claim 9, wherein the at least one range finder comprises two or more range finders, and wherein the two or more range finders measure the distance between the depth sensing drill chuck and the target surface to generate multiple measured distances, and wherein the multiple measured distances are averaged to overcome variances due to uneven variations in the target surface or due to changes in the rotary position of a drill body.

14. The depth sensing drill chuck of claim 13, further comprising two or more laser aiming motors that keeps the two or more lasers focused on two or more focus spots on the target surface as the depth sensing drill chuck moves closer to the target surface during drilling.

15. The depth sensing drill chuck of claim 9, wherein the one or more range finder is located on the rotary portion so that the distance between the depth sensing drill chuck and the target surface can be measured at multiple locations around the 360-degree rotary path.

16. The depth sensing drill chuck of claim 13, further comprising a laser aiming motor that keeps the laser focused on a focus spot on the target surface as the depth sensing drill chuck moves closer to the target surface during drilling.

17. A method of fixing broken bones comprising:
drilling a hole in a bone using a depth measuring drill chuck, the depth measuring drill chuck comprising a load cell and at least one range finder adapted to measure a distance between the depth sensing drill chuck and the broken bone, the depth measuring drill chuck calculating a penetration depth in real time while drilling the hole; and
reading the penetration depth from a display screen.

18. The method of claim 17, wherein reading the penetration depth from the display screen further comprises reading the penetration depth from the display screen in real time, while drilling.

19. The method of claim 17, further comprising selecting a fixing screw with a length that matches the penetration depth, and further comprising screwing the fixing screw into the hole, wherein the screw length is the same as the hole depth.
